(19) United States
(12) Patent Application Publication
(10) Pub. No.: US 2024/0407858 A1
(43) Pub. Date: Dec. 12, 2024

(54) NAVIGATED INSTRUMENT FOR USE IN ROBOTIC GUIDED SURGERY

(71) Applicant: GLOBUS MEDICAL, INC., AUDUBON, PA (US)

(72) Inventor: Neil Crawford, Chandler, AZ (US)

(21) Appl. No.: 18/809,450

(22) Filed: Aug. 20, 2024

Related U.S. Application Data
(63) Continuation of application No. 17/120,879, filed on Dec. 14, 2020, now Pat. No. 12,064,189.
(60) Provisional application No. 62/947,688, filed on Dec. 13, 2019.

(51) Int. Cl. A61B 34/20; A61B 34/30; A61B 90/00

(52) U.S. Cl. CPC A61B 34/20; A61B 34/30; A61B 90/39; A61B 2034/2055

(57) ABSTRACT

An instrument for use in a navigated surgical procedure, the instrument includes a proximal portion, a distal portion and a shaft extending therebetween. An angled instrument tip is positioned at an end of the distal portion of the instrument. A first tracking array is coupled to the proximal portion of the instrument and a surveillance array is coupled to the proximal portion of the instrument. The tracking array includes a plurality of tracking markers, and is configured to rotate with respect to a central axis of the instrument.

### CROSS REFERENCE TO RELATED APPLICATIONS

This application is a continuation of U.S. patent application Ser. No. 17/120,879 filed on Dec. 14, 2020, which claims priority to provisional application Ser. No. 62/947,688 filed on Dec. 13, 2019, all of which are incorporated in their entirety herein.

### FIELD OF THE INVENTION

The present disclosure relates to medical devices, and more particularly to an instrument for use with a robotic surgical system.

### BACKGROUND

Position recognition systems for robot assisted surgeries are used to determine the position of and track a particular object in 3-dimensions (3D). In robot assisted surgeries, for example, certain objects, such as surgical instruments, need to be tracked with a high degree of precision as the instrument is being positioned and moved by a robot or by a physician, for example.

Position recognition systems may use passive and/or active sensors or markers for tracking the objects. In passive sensors or markers, objects to be tracked may include passive sensors, such as reflective spherical balls, which are positioned at strategic locations on the object to be tracked. With passive tracking sensors, the system then geometrically resolves the 3-dimensional position of the active and/or passive sensors based on information from or with respect to one or more of the infrared cameras, digital signals, known locations of the active or passive sensors, distance, the time it took to receive the responsive signals, other known variables, or a combination thereof.

These surgical systems can therefore utilize position feedback to precisely guide movement of robotic arms and tools relative to a patients' surgical site. In surgical navigation, commonly tracked tools like a drill, tap or screwdriver are typically axially symmetrical. For example, a representation of a drill looks the same no matter how the drill bit is rotated. The tracking array for such tools may be mobile in its rotational coordinate about the tool since the rotational position of the tool does not need to be monitored. Therefore, marker arrays for tracking these common tools are designed with the array on a sleeve that is free to rotate about the tool. The user may reposition the array about the tool shaft as necessary to keep it facing toward the tracking cameras while using the tool.

However, it is sometimes necessary to track a tool that is not symmetrical, such as a curved curette or a delivery device for an interbody spacer. In such cases, the system must track the full rigid body position of the tool so that it can properly update the image of the tool overlaid on anatomy, showing, for example, which direction the curve or cutting surface of the curette faces. For these tools and instruments, different methods must be used to find the tracking array's orientation relative to the tool in all directions including rotation.

### SUMMARY

An instrument for use in a navigated surgical procedure, the instrument includes a proximal portion, a distal portion and a shaft extending therebetween. An angled instrument tip is positioned at an end of the distal portion of the instrument. A first tracking array is coupled to the proximal portion of the instrument and a surveillance array is coupled to the proximal portion of the instrument. The tracking array includes a plurality of tracking markers, and is configured to rotate with respect to a central axis of the instrument.

### BRIEF DESCRIPTION OF THE DRAWINGS

The accompanying drawings, which are included to provide a further understanding of the disclosure and are incorporated in a constitute a part of this application, illustrate certain non-limiting embodiments of inventive concepts. In the drawings:

FIGS. 1A, 1B and 1C are views of a surgical instrument according to one embodiment of the present invention.

FIGS. 2A and 2B illustrates an embodiment of a surgical instrument with an array having adjustable positions.

FIGS. 3A and 3B illustrates the surgical instrument with centerline markers and offset markers for use with a surgical robotic system according to some embodiments;

FIG. 4 illustrates another embodiment of the surgical instrument having multiple offset markers and center line markers.

FIGS. 5A, 5B, 5C, and 5D illustrate yet another embodiment of a surgical instrument that allows rotation of an array by use of a cam mechanism.

FIGS. 6A, 6B, 6C, and 6D show another embodiment of a surgical instrument that uses a hinged cam mechanism to allow rotation of an array.

FIGS. 7A and 7B illustrate another embodiment of a surgical instrument that uses multiple cam mechanisms for rotation of an array.

FIGS. 8A-8D illustrates yet another embodiment of a surgical instrument according to the present disclosure.

FIG. 9 illustrates another embodiment of a surgical instrument with a plurality of tracker arrays.

FIG. 10 illustrates yet another embodiment of a surgical instrument in which the array is configured to swivel about central axis of the surgical instrument.

### DETAILED DESCRIPTION

It is to be understood that the present disclosure is not limited in its application to the details of construction and the arrangement of components set forth in the description herein or illustrated in the drawings. The teachings of the present disclosure may be used and practiced in other embodiments and practiced or carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following discussion is presented to enable a person skilled in the art to make and use embodiments of the present disclosure. Various modifications to the illustrated embodiments will be readily apparent to those skilled in the art, and the principles herein can be applied to other embodiments and applications without departing from embodiments of the present disclosure. Thus, the embodiments are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of the embodiments. Skilled artisans will recognize the examples provided herein have many useful alternatives and fall within the scope of the embodiments.

In surgical navigation, it is very important that a tool's tracker marker template be accurate so that a tracked tool and/or tracked robot end effector is correctly localized relative to the anatomy. A method is whereby a sequence of simple and rapid test movements using a navigated tool and a tracked rigid guide tube, such as the guide tube of a surgical robot, can fully calibrate a tool that was initially not calibrated. The calibrated tool will have its shaft coincident with one coordinate axis, the markers facing toward a pre-defined direction, and the origin offset by a pre-defined distance from the origin of the tracked rigid guide tube.

Surgical navigation uses an optical tracking system where two or more cameras detect reflective or active markers on tools and track the 3D coordinates of the markers through stereophotogrammetry. As discussed in the previous embodiments, surgical tools are equipped with rigidly mounted arrays of 3 or more reflective or active markers, and the tracking system uses best-fit algorithms to match the detected marker locations to stored templates of ideal marker locations. In this way, the system can determine which tool or tools are in view, and where the tools, as rigid bodies, are located in the coordinate system of the cameras. Virtual landmarks can also be found. For example, from the tracked locations of markers that are mounted on a tool's handle, the system finds the location of the tool shaft and tip in camera space. Once tool locations are known relative to the cameras and registration has been performed to align the coordinate system of the cameras to the coordinate system of the medical image, it is possible to graphically display tool locations overlaid on the medical images.

Several factors can influence the accuracy of surgical navigation. The array of optical markers that is attached to the tool is poorly defined or can become displaced, either in whole or in part, causing errors because the detected markers positions are shifted or do not fit the stored marker template well. Other factors included inconsistent lighting and poor viewing angles of markers. In surgical navigation, errors in tracking of poorly-calibrated navigated tools can be manifested as “wander” of the graphic of the tool when the user rotates the tool within a rigid tube. That is, the display of a tool that is rotated within a rigid tube should not move since the tool is spinning about its axis without translating or bending. If the display does in fact wander, it confirms that the tracked location of the tool's array does not match the stored template for the tool.

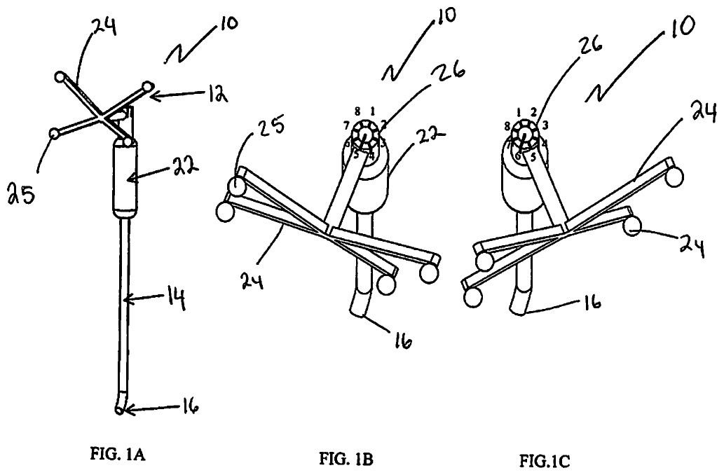

Now turning to FIGS. 1A-1C, there is shown a tracking array coupled to an instrument that optimally allows the tracking of the instrument according to one embodiment. When a tracking array is rigidly mounted to a tool, the array is designed so that it is pointing optimally toward the cameras while the tool is held in the most often used position.

FIGS. 1A, 1B, and 1C shows a navigated tool 10 having a proximal portion 12, a middle portion 14, and a distal portion 16. The distal portion 16 of the navigated tool 10 in this embodiment is provided with a curved tip 18. A shaft 20 extends from the curved tip 18 to the proximal portion 12. At the proximal portion of the tool 18, there is a handle 22 and a tracking array 24. The tracking array 24 is configured to be rotated with respect to the longitudinal axis of the navigated tool 10. The adjustable tracking array 24 also includes a calibration dial 26 positioned on an upper surface of the tool 10. The tracking array 24 is also configured to be positioned at any rotational position and may be locked into position and identified by a locking position as marked on the calibration dial 26. When the tracking array 24 is locked in a particular orientation relative to the handle 22, the user may enter or the system detects the rotational coordinate and the system shows a representation of the tool 10 overlaid on the anatomy. As shown in FIG. 1B and FIG. 1C, the tracking array 10 is locked in position 5 (center) or position 6 (right) to allow tracking cameras to maintain a line of sight for tracking the full motion of the tool 10. The tracking array 24 in the present embodiment illustrates a plurality of markers 25. It should be noted that in other embodiments, it is contemplated that the tracker array 24 may having a more than additional or less markers than shown in the current embodiment. The tracker array 24 may also be configured as a different shape than illustrate in FIGS. 1A-1C.

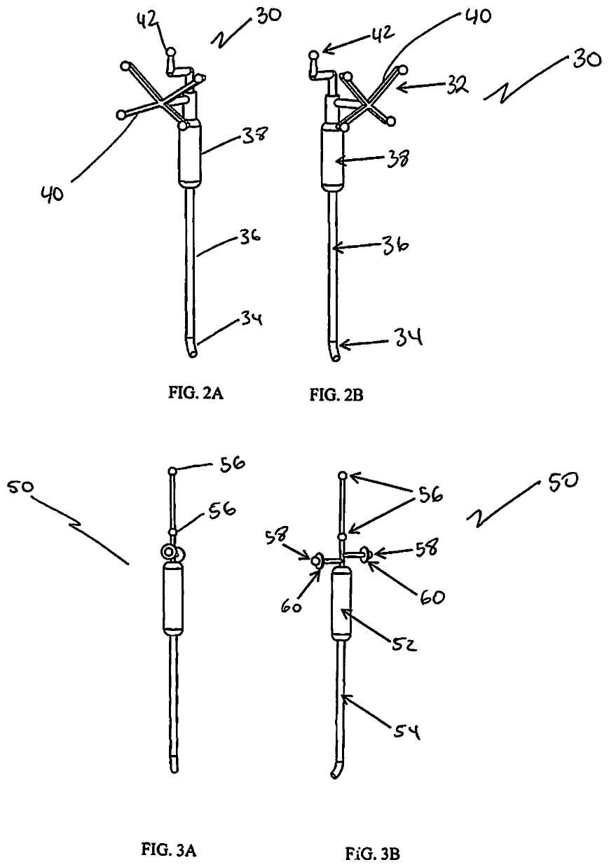

In another embodiment as shown in FIGS. 2A and 2B, a multi-positional array system 30 includes a proximal portion 32, a distal portion 34 and a shaft 36 extending from the proximal portion to the distal portion 34. The distal portion 34 includes a curved tip in one embodiment, but include any type of tip that is suitable for a surgical procedure. The proximal portion also includes a handle 38 and a rotatable tracking array 40, and a fixed marker 42. The fixed marker 42 may be used as an additional tracking marker as a means of automatically identifying the system 30 position relative to the tracking array 40. This additional fixed marker 42 must be offset from the center of rotation of the tracking array (center shaft of the tool) to detect array position.

FIGS. 2A and 2B also shows an embodiment of an array 40 that is rotationally adjustable. The tool array 40 is rotatably movable relative to the handle 38 and shaft 36. A single fixed marker 42 mounted to the central shaft detects the rotational position of the tracking array relative to the shaft, allowing correct tracking and graphical representation of the entire tool including the curved tool tip. Although in the present embodiment the fixed marker 42 is shown in a particular offset location, it is contemplated in other embodiments, that the fixed marker 42 may be configured in type of offset position relative to the tool and tracking array. Also, it the present embodiment, the tracking marker 42 is positioned in a offset position from the upper surface of the tool, it is contemplated in other embodiments that the offset tracking marker 42 may be positioned laterally with respect to the tool 30.

Now turning to FIGS. 3A and 3B, an instrument 50 having a handle 52, a shaft 54 and a plurality of tracking markers is shown. The instrument 50 includes at least two centerline markers 56 and at least two offset markers 58. The offset markers 58 extend a distance away from the central axis of the instrument 50. The offset markers 58 are also provided with shields 60. The centerline markers 56 and offset markers 58 are rigidly attached to the instrument 50. The centerline markers 56 and offset markers 58 are provided with features allowing it to be viewed from a wide range of angles. The offset markers 58 are at different longitudinal coordinates along the centerline, allowing the system to distinguish between the offset markers 58 when only one is visible by the distance from the centerline markers 56. Alternately, the offset markers 58 could be at different radial distances offset from centerline but at the same longitudinal coordinate to allow them to be distinguished. The thin flat shield 60 behind each offset marker hides the marker from view and prevents visual overlap with other markers or parts of the tool when the tool is rotated away from the cameras. Although in the present embodiment, only two offset markers and centerline makers are shown, it is contemplated that a plurality of either centerline and/or offset makers may be utilized to determine the position of the instrument.

In another embodiment, a part of one offset marker may be visible farther from the cameras due to partial obstruction from its shield and the whole of another offset marker is visible closer to the cameras. The system can utilize the larger detected 2D “blob” (high contrast region) size in deciding which of the two offset markers to use in calculations to get the most accurate navigation. Alternately, the system may also use calculations for both sets of 3 markers (two centerline markers plus offset marker 1 or two centerline markers plus offset marker 2) to determine the tip location of the instrument.

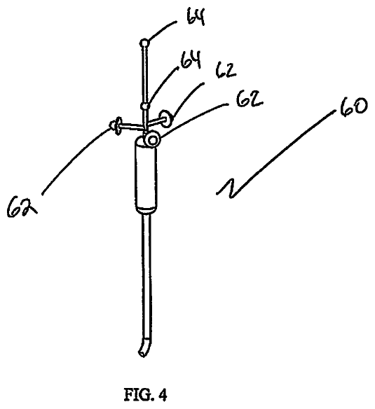

In another embodiment as shown in FIG. 4, there is provided an instrument 60 that utilizes three offset markers 62 at 120° separation, ensuring that at least one of the offset markers is fully visible to both cameras at any orientation. The instrument 60 also includes at least two colinear markers 64 positioned on the central axis of the instrument.

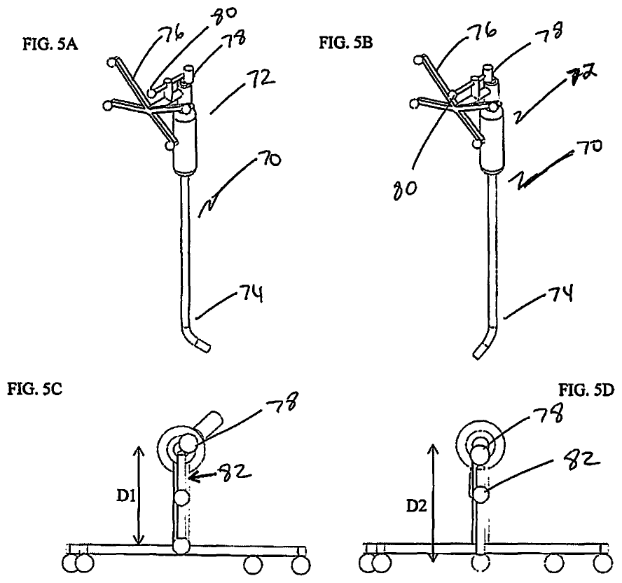

FIGS. 5A and 5B shows an instrument 70 with a proximal portion 72 and a distal portion 74. The proximal portion includes a tracking array 76 and a single cam mechanism 78 that causes an tracked marker 80, which is positioned on a spring-loaded piston 82, to move toward or away from a central shaft of the instrument 70 when the tracking array 76 is rotated about the shaft. The cam mechanism 78 is an offset circular or oblong shaped element on which a spring-loaded plunger 82 presses. This mechanism 78 causes the plunger 82 to move inward and outward by varying amounts as the array orientation is adjusted relative to the instrument tip as shown in FIG. 5C and FIG. 5D. After calibrating the angle of the tracker array relative to marker's 80 linear position, by tracking the offset of the additional marker, the system can determine the orientation of the instrument 70. As the instrument tip is moved and angled as shown as about 135 degrees counterclockwise as shown in FIG. 5C, the marker 80 is offset to position D1. If the instrument tip is angled to about 5 degrees clockwise as shown in FIG. 5D, the marker 80 is moved to offset position D2. As shown in FIGS. 5C and 5D, the cam mechanism 78 is configured to provide information to the tracking system about the orientation of the tip relative to the instrument. In this embodiment, the tracker array 76 is provided with 4 markers, it is contemplated in other embodiments, additional or less markers may be used to navigate the instrument.

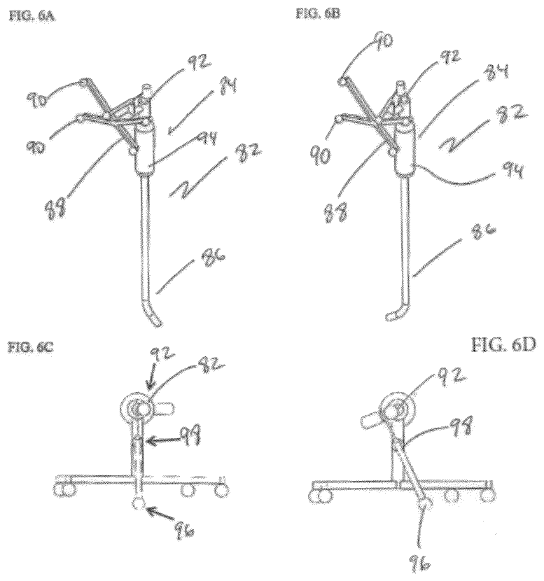

FIGS. 6A-6D show an instrument 82 having a proximal portion 84 and a distal portion 86. The proximal portion 84 includes a tracker array 88 that includes a plurality of trackers 90. The proximal portion 84 also includes a single cam mechanism 92 coupled to a handle assembly 94 of the instrument 82. The single cam mechanism 92 is coupled to the upper surface of the handle assembly 94 of the instrument. The cam mechanism 92 is also coupled to a tracking marker 96. Tracking marker 96 is positioned on a hinged, spring-loaded arm 98 and configured to move toward or away from the central shaft of the instrument when the tracker array 88 is rotated about the shaft. As further illustrated in FIGS. 6C and 6D, the spring loaded arm 98 is configured to swivel in varying directions allowing for the tracked marker 96 to be visualized by the camera system at different locations. After calibrating angle of tracker array 88 relative to tracker marker 96 position, by detecting the offset of the marker 96, the system can determine the orientation of the instrument.

With either a linear or hinged mechanism, the moving position of the tracker marker can be detected relative to the positions of the rest of the markers in the tracking array and this information used to determine the orientation of the instrument tip relative to the tracking array. Then, as the surgeon uses the instrument, the tracker array may face toward the cameras while positioning the instrument appropriately to perform surgical work, and the system can continuously track the instrument position while also showing a correct graphical representation of the instrument, including its asymmetrical tip.

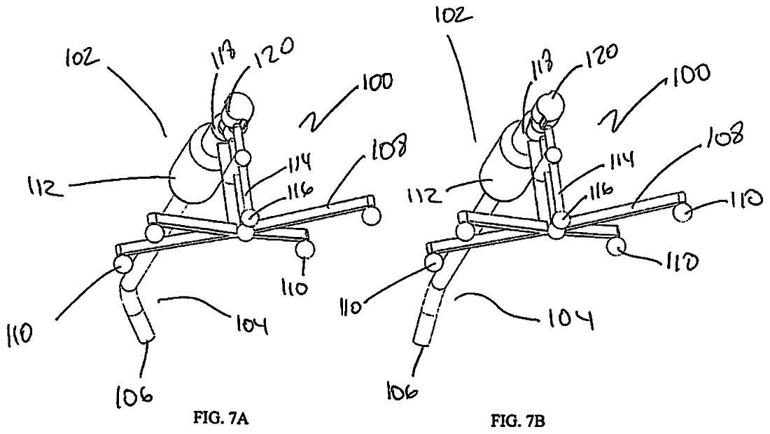

Now turning to FIGS. 7A and 7B, a system for unambiguously defining the instrument tip orientation of an instrument through a full 360 degree range using two cam mechanisms is shown. As show in FIGS. 7A and 7B, a surgical instrument 100 having a proximal portion 102 and a distal portion 104. The distal portion 104 is provided with an angled end or tip 106 for performing surgical procedures. The proximal portion 102 is provided with a first tracking array 108. The first tracking array 108 is provided with a plurality of tracking markers 110. Each of the plurality of tracking makers 110 are spaced apart from one another and configured to be detected by a camera system. The proximal portion 102 of the instrument 100 also includes a handle assembly 112. The proximal portion 102 also includes a second tracker array 114 having at least one tracker marker 116. The second tracker array 114 is coupled to the proximal portion 102 via a first cam mechanism 118 and a second cam mechanism 120. The first cam mechanism 118 is configured to enable the second tracker array 114 to moved vertically with respect to the longitudinal axis of the instrument 100. The second cam mechanism 120 is configured to enable the second tracker array 114 to be moved horizontally with respect to the longitudinal axis of the instrument 100. As each cam mechanism controls the position of tracking marker 116, the positions of the marker 116 can be identified relative to the tracking array 108.

In another embodiment, the second cam mechanism may be configured to shift the whole assembly of the first cam mechanism, thereby, the first cam mechanism may shift the marker 116 inward and outward along a piston toward or away from the tracker array 108, whereas the second cam mechanism could shift the marker 116 longitudinally up or down in the direction of the instrument relative to the tracker array 108.

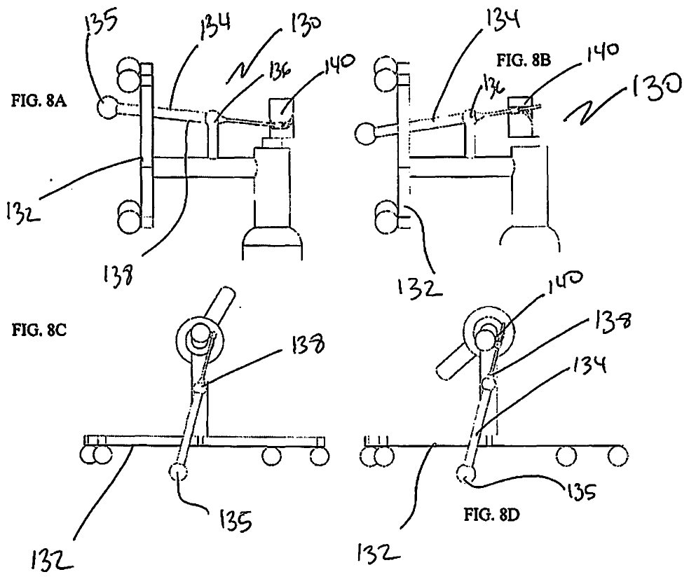

Now turning to FIGS. 8A-8D, another embodiment for determining the rotational positioning of an instrument 130 is provided. FIGS. 8A-8D illustrates a proximal portion of the instrument 130. The instrument 130 includes a first tracker array 132 and a second tracker array 134 with a tracker marker 135. Tracker marker 135 is attached to a hinged cam assembly 136. The hinged cam assembly 136 includes a lever arm 138 attached to the cam mechanism 140. As the instrument is rotated, the tracker marker 135 is moved rotationally and angularly with respect to the central longitudinal axis of the instrument 130. As a result, the tracker marker 135 along with the first tracker array 132, is tracked by the camera system allowing the instrument tip to accurately tracked during surgical procedures.

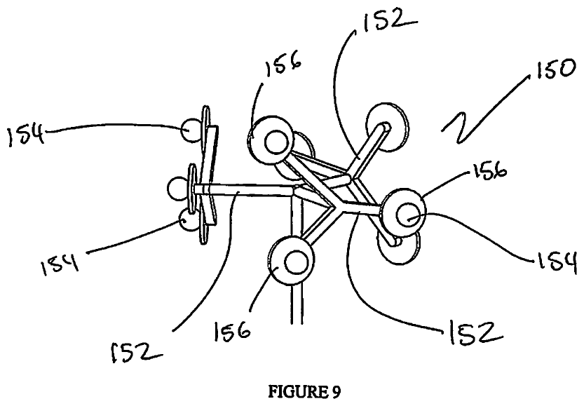

In yet another embodiment, FIG. 9 illustrates an instrument 150 having multiple tracking arrays 152. Each of the tracking arrays 152 is configured with a plurality of tracking markers 154, with each tracking marker 154 having a shield 156. The tracking arrays 152 are deployed about the center axis of the instrument 150. In one embodiment, the system would select for tracking only the 3 or more markers closest to the cameras and in best view of the cameras. The tracking arrays 152 in this embodiment provide 3 markers each however it is contemplated in other embodiments to have additional or less markers for each tracking array 152. The tracking arrays 152 are also configured to be detected by a camera system from all angles.

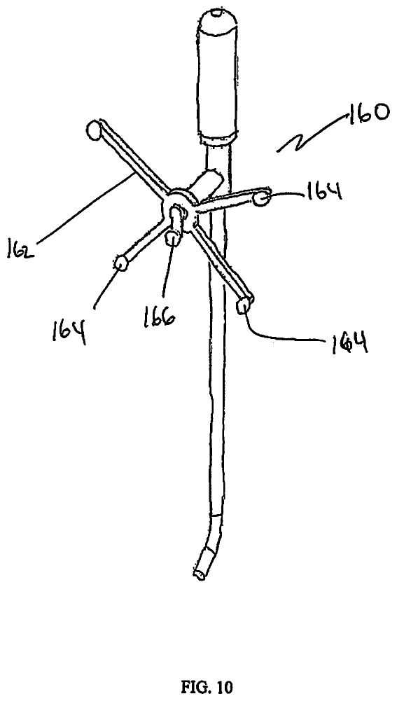

FIG. 10 shows an embodiment in which surgical instrument 160 includes a tracker array 162 that is capable of rotating with respect to the longitudinal axis of the instrument. The tracker array 162 includes plurality of markers 164 and a surveillance marker 166 positioned on a central portion of the tracker array 162. The surveillance marker 166 is configured to rotated with respect to the tracker array 162 and is configured to identify the array position relative to the instrument tip. The surveillance marker 166 is mounted directly to the tracker array 162 face on the central portion of the tracker array 162. In other embodiments, it is contemplated that the surveillance marker 166 is configured to rotated and translated with respect to the instrument 160.

Now turning to FIG. 11, a tracking system and method of performing a procedure to optimize the tracking of markers is provided. When an optical tracking system tracks the markers on a tool or instrument, it performs a comparison of the detected markers to the known stored marker template for that tool or instrument using computational methods. Other important landmarks of the tool or instrument such as its tip and tail are designed to be in known locations in the template coordinate system. During any tracking frame, using computational methods, the system transforms the coordinates of any landmark defined within the template coordinate system to camera space based on the tool's detected markers in that frame.

In one embodiment of the invention, the positioning of physical features of the tool within the coordinate system of the marker template can serve to re-define the tool template during a simple calibration procedure. These features of the tool are defined such that it is exactly aligned with and positioned along the Cartesian coordinate (X, Y, Z) axes according to standards used for registration. The navigated tool or instrument includes a shaft that is aligned along the Z axis, with a tail that is configured to be in a more positive position than the tip of the tool or instrument. The marker array coupled to the instrument is aligned at a rotational position about the Z axis at which the marker's plane normal is aligned in a X direction. The tool or instrument is positioned longitudinally along the Z axis with its Z origin (Z=0) at the location where the tool bottoms out on the guide tube.

Using a tool or instrument as provided, the following sequences may be applied in one embodiment of the invention. The system may record the end effector markers or calibration tool markers to establish where an inserted tool bottoms out relative to the end effector guide tube 170. Next, the navigated tool is inserted until it bottoms out in the tube. Then the tool or instrument may be swiveled back and forth about its shaft between 45-90° while keeping the tool or instrument bottomed out.

The data is recorded from these steps in tracking the instrument and used to create an updated tool or instrument template file using the following algorithm. The helical axis of motion in the camera space from the frames of data recoding the swiveling of the instrument is recoded. The axis represents the functional center of the tool or instrument shaft. Next, the system transforms the helical axis into alignment with the Z-axis and these transformations are applied to each data set. Then the instrument or tool is translated along the Z-axis unit it bottoms out and matches the bottom out point of the end effector. Next, the tool is rotated to position the marker array in the positive X direction. After these transformations are applied to the point set, the marker positions in the tool template file are replaced with the marker position in the new coordinate system.

It is also contemplated that software may “repair” the calibration of a tool if it is discovered during usage that the calibration is off. In one embodiment, the user would indicate to software that a repair is being initiated, possibly by pressing a button or otherwise activating a feature that sets a flag indicating that the repair should occur on the next tool coming into close proximity with the end effector guide tube. When a tool is bottomed out in the guide tube, the origin of the tool and the origin of the guide tube should be nearly coincident if the guide tube's origin is also defined as the tube's center and upper rim, or the point where any tool entering it bottoms out.

In the first step of a repair workflow, it is noted that it should be specified which markers belong to the tool's array. Providing the current (damaged) template for the purpose of sorting and matching incoming markers is one possible way to specify the desired markers since each frame can be compared to the damaged template and only markers that are within a tolerance of matching the template pattern can be automatically selected. Alternately, a software interface could take a static shot and markers belonging to the tool could be manually marked on a software interface; software monitors and tracks the markers as they move. In another embodiment, the system may utilize elements that strobe in a known pattern, allowing markers to be indexed and always stored in the same order for each frame. In such systems, the list indices of markers belonging to the tool could be specified.

In later steps, the helical axis of motion (HAM) is calculated. Computational methods used for calculating the finite HAM or approximating the instantaneous HAM from tracked marker data are applied, and better accuracy is achieved with larger angular step sizes.

In some embodiments, instead of repairing an existing template, the swivel method can be used to successfully create an accurate tool marker template without any starting template. Therefore, such a method could construct an accurate marker template for a tool with markers arbitrarily glued, bolted or otherwise secured to its tool shaft as long as the tool bottoms out at a known position and has a central shaft that can be swiveled. If the length of a tool from the location where it bottoms out (its origin) to the tip and the tool's shaft diameter is provided, then all of the critical information about the tool for safe navigation is specified. A generic tool appearing overlaid on the anatomy as a cylinder of specified diameter with the tip offset at the specified distance from the origin could be overlaid on the anatomy to represent the tool and accurately visualize the anatomy intersected by the tool.

Incorporating various methods of tracking the array as well as asymmetric tip orientation and allowing the updating of the CAD model in the software is more accurate in two main orientations, and depending on array pattern in respect to the camera, allows the surgeon to track in a wider variety of positions. Tracking such as this is especially helpful during tracking where the CAD model orientation updates automatically without UI input from the surgeon.

In the above-description of various embodiments of present inventive concepts, it is to be understood that the terminology used herein is for the purpose of describing particular embodiments only and is not intended to be limiting of present inventive concepts. Unless otherwise defined, all terms (including technical and scientific terms) used herein have the same meaning as commonly understood by one of ordinary skill in the art to which present inventive concepts belong. It will be further understood that terms, such as those defined in commonly used dictionaries, should be interpreted as having a meaning that is consistent with their meaning in the context of this specification and the relevant art and will not be interpreted in an idealized or overly formal sense unless expressly so defined herein.

When an element is referred to as being “connected”, “coupled”, “responsive”, or variants thereof to another element, it can be directly connected, coupled, or responsive to the other element or intervening elements may be present. In contrast, when an element is referred to as being “directly connected”, “directly coupled”, “directly responsive”, or variants thereof to another element, there are no intervening elements present. Like numbers refer to like elements throughout. Furthermore, “coupled”, “connected”, “responsive”, or variants thereof as used herein may include wirelessly coupled, connected, or responsive. As used herein, the singular forms “a”, “an” and “the” are intended to include the plural forms as well, unless the context clearly indicates otherwise. Well-known functions or constructions may not be described in detail for brevity and/or clarity. The term “and/or” includes any and all combinations of one or more of the associated listed items.

It will be understood that although the terms first, second, third, etc. may be used herein to describe various elements/operations, these elements/operations should not be limited by these terms. These terms are only used to distinguish one element/operation from another element/operation. Thus a first element/operation in some embodiments could be termed a second element/operation in other embodiments without departing from the teachings of present inventive concepts. The same reference numerals or the same reference designators denote the same or similar elements throughout the specification.

As used herein, the terms “comprise”, “comprising”, “comprises”, “include”, “including”, “includes”, “have”, “has”, “having”, or variants thereof are open-ended, and include one or more stated features, integers, elements, steps, components or functions but does not preclude the presence or addition of one or more other features, integers, elements, steps, components, functions or groups thereof. Furthermore, as used herein, the common abbreviation “e.g.”, which derives from the Latin phrase “exempli gratia,” may be used to introduce or specify a general example or examples of a previously mentioned item, and is not intended to be limiting of such item.

Although several embodiments of inventive concepts have been disclosed in the foregoing specification, it is understood that many modifications and other embodiments of inventive concepts will come to mind to which inventive concepts pertain, having the benefit of teachings presented in the foregoing description and associated drawings. It is thus understood that inventive concepts are not limited to the specific embodiments disclosed hereinabove, and that many modifications and other embodiments are intended to be included within the scope of the appended claims. It is further envisioned that features from one embodiment may be combined or used with the features from a different embodiment(s) described herein. Moreover, although specific terms are employed herein, as well as in the claims which follow, they are used only in a generic and descriptive sense, and not for the purposes of limiting the described inventive concepts, nor the claims which follow. The entire disclosure of each patent and patent publication cited herein is incorporated by reference herein in its entirety, as if each such patent or publication were individually incorporated by reference herein. Various features and/or potential advantages of inventive concepts are set forth in the following claims.

We claim:

1. A method of determining an orientation of an instrument in a navigated surgical procedure, the method comprising:
providing a navigated instrument including:
a shaft defining a central axis;
a working piece attached to the shaft and extending radially outwardly from the shaft;
a tracking array rotatably attached to the shaft for rotation about the central axis and including a plurality of tracking markers trackable by a tracking device;
a surveillance marker coupled to the instrument and trackable by the tracking device; and
determining a circumferential position of the tracking array relative to the working piece based on the position of the surveillance marker and the tracking array as received from the tracking device.

2. The method of claim 1, wherein determining a circumferential position of the tracking array includes determining 3D coordinates of the tracking markers.

3. The method of claim 2, wherein the plurality of tracking markers are optical markers trackable by cameras of the tracking device through stereophotogrammetry.

4. The method of claim 1, wherein the tracking array is configured to rotated 360 degrees about the central axis of the instrument.

5. The method of claim 1, wherein the surveillance marker includes a plurality of surveillance markers positioned apart from the tracking array.

6. The method of claim 1, wherein the surveillance marker includes at least two centerline markers positioned on the central axis of the instrument and at least two offset markers offset laterally with respect to the central axis of the instrument.

7. The method of claim 1, wherein the surveillance marker is configured to be radially movable with respect to the central axis of the instrument.

8. The method of claim 1, wherein determining a circumferential position of the tracking array includes determining 3D coordinates of the tracking markers by the tracking device associated with a surgical robot.

9. The method of claim 1, wherein the surveillance marker is configured to be swivelable at an angle with respect to the central axis of the instrument.

10. A method of determining an orientation of an instrument in a navigated surgical procedure, the method comprising:
providing a navigated instrument including:
a shaft defining a central axis and having a proximal portion and a distal portion;
a working piece attached to the distal portion of the shaft and extending radially outwardly from the shaft;
a tracking array rotatably attached to the proximal portion of the shaft for rotation about the central axis and including a plurality of tracking markers trackable by a tracking device;
a rotatable and translatable surveillance marker coupled to the proximal portion of the instrument and trackable by the tracking device; and
determining a circumferential position of the tracking array relative to the working piece and the central axis based on the position of the surveillance marker and the tracking array as received from the tracking device.

11. The method of claim 10, wherein determining a circumferential position of the tracking array includes determining 3D coordinates of the tracking markers.

12. The method of claim 11, wherein the plurality of tracking markers are optical markers trackable by cameras of the tracking device through stereophotogrammetry.

12. The method of claim 10, wherein the tracking array is configured to rotated 360 degrees with respect to the central axis of the instrument.

13. The method of claim 10, wherein the surveillance marker includes a plurality of surveillance markers trackable by the tracking device.

14. The method of claim 10, wherein the surveillance marker is spaced from the tracking array.

15. The method of claim 10, wherein the surveillance marker is configured to be movable radially outwardly and linearly with respect to the central axis of the instrument.

16. The method of claim 10, wherein determining a circumferential position of the tracking array includes determining 3D coordinates of the tracking markers by the tracking device associated with a surgical robot.

17. The method of claim 10, wherein the surveillance marker is configured to be swivelable at an angle with respect to the central axis of the instrument.
